(19) United States
(12) Patent Application Publication
(10) Pub. No.: US 2019/0192909 A1
(43) Pub. Date: Jun. 27, 2019

(54) SYSTEMS AND METHODS FOR PERSONAL FITNESS

(71) Applicant: Northwest Rehabilitation and Wellness, LLC, Salem, OR (US)

(72) Inventors: Michael Todd Studer, Monmouth, OR (US); Robert Gunder Winningham, Salem, OR (US)

(21) Appl. No.: 16/231,025

(22) Filed: Dec. 21, 2018

Related U.S. Application Data
(60) Provisional application No. 62/610,229, filed on Dec. 24, 2017.

(51) Int. Cl. A63B 24/00; G16H 50/30; G16H 20/30

(52) U.S. Cl. CPC A63B 24/0062; A63B 24/0075; A63B 2024/0068; G16H 20/30; A63B 2024/0065; G16H 50/30

(57) ABSTRACT

A system can include a sensor configured to obtain multiple measurements from a user following a prescribed exercise regimen, and a processor configured to calculate dual task cost physical and cognitive benchmarks based on the measurements and determine whether to change the prescribed exercise regimen based on the calculated dual task cost physical and cognitive benchmarks.

### CROSS REFERENCE TO RELATED APPLICATION

This application is a non-provisional of and claims benefit from U.S. Provisional Patent Application Ser. No. 62/610,229, filed on Dec. 24, 2017 and entitled METHOD FOR USING DUAL TASK FITNESS, the entire content of which is hereby incorporated by reference herein.

### TECHNICAL FIELD

The disclosed technology is generally directed to systems and methods for personal fitness assessment and treatment. More particularly, implementations generally pertain to integrating physical and cognitive fitness into dual task fitness assessment and treatment.

### BACKGROUND

The assessment of dual task performance, e.g., concurrent performance of two tasks with distinct and separate goals, is an emerging concept in the world of medicine and more specifically in rehabilitative medicine where the testing provides insight into the capacities of individuals with cognitive impairment to function in a world filled with distractions. Cognitive testing in isolation from real-world distractions may not provide a valid predictor of future outcomes. Currently, it is usually unclear how cognitive or physical impairments translate to higher level athletic or even functional day-to-day activities and safety concerns. Dual task training can thus reveal difficulties in the integration of higher demands (e.g., physical and cognitive) for a wide range of applications. This methodology can provide insight for athletes performing at high skill levels, with superimposed pressures and distractions of the sport, or for people that can otherwise compensate for or cover up their discrete physical or cognitive impairments when only cognitive or physical impairment is measured.

Wellness and health of all kinds (e.g., physical, cognitive, spiritual) have become increasingly important to those without known health conditions or comorbidities. Cognitive fitness and training have only recently been exposed to randomized clinical trials. In addition, the proliferation of commercial products to promote cognitive fitness is even more recent. Those with degenerative and one-time neurologic conditions such as Parkinson's, multiple sclerosis, stroke, etc. are recognizing the benefits of physical and cognitive wellness for fitness and fall prevention, as seen in both the consumer and research arenas.

Within physical fitness, the concept of High Intensity Training (HIT) has proven to be effective in strength and endurance training, through separate but related applications. The benefits of HIT have been experimentally proven to include stimulating equivalent levels of improvements with less time and less exposure to injury in training through fewer repetitions to achieve similar gains. HIT generally includes any schedule of higher intensity (e.g., effort) represented in any quantifiable parameter of performance requiring a greater effort to achieve. These higher periods are interspersed in the form of regular or irregular (e.g., surprise or unscheduled) periods of lower effort. Quantifiable performance indicators can include all recordable data from the workout, including but not limited to perceived exertion, speed, incline, revolutions per minute (RPM), watts, metabolic equivalents (METs), calories, distance traveled, steps per minute, stride length, symmetry, weight bearing, step length, and ground reaction forces. These performance indicators can be captured by way of fitness equipment, activity monitors, or personal recording efforts (e.g., walking distance, hand weight repetitions, etc.).

Previously, users simply tested performance in either cognitive or physical single task performance and then tried to subsequently improve their cognitive or physical single task performance.

Implementations of the disclosed technology address these and other limitations in the prior art.

### BRIEF DESCRIPTION OF THE DRAWINGS

Aspects, features and advantages of embodiments of the present disclosure will become apparent from the following description of embodiments in reference to the appended drawings in which:

FIG. 1 illustrates an example of a method of using dual task fitness in accordance with certain implementations of the disclosed technology.

FIG. 2 illustrates an example of a system for using dual task fitness in accordance with certain implementations of the disclosed technology.

### DESCRIPTION

Certain implementations may include single-task testing using various parameters for physical performance that can be applied for baseline single task physical fitness. After these original baseline single task measurements of performance are measured, the user can engage in high intensity dual task training and a high intensity dual task cost can be calculated for cognition and physical fitness. Based on the dual task costs, an algorithm can be used to determine whether the user should progress to higher or lower levels of high intensity dual task training intensity (e.g., more challenging cognitive tasks and/or more challenging physical tasks).

The progression of training can be turned into a game by allowing the user to earn points and/or progress to higher numerical levels. Dual Task High Intensity Training as described herein generally includes the integration of Dual Task Training and High Intensity Training. This combination combines cognitive fitness and physical fitness and can be used to customize the prescription using baseline single task testing (e.g., cognitive testing and physical testing).

In certain embodiments, the dual task training methodology can include a first step of measuring a person's cognitive and physical dual task abilities while performing high intensity training, and a second step of prescribing a cognitive or physical treatment by using a person's cognitive and/or physical dual task abilities. Additional steps can be added to improve performance of the methodology. The steps may be done one or more times, or a series of steps may be done in sequence repeatedly before moving to the next step.

In certain embodiments, the Dual Task High Intensity Training (DT HIT) methodology can include a first step of measuring a person's maximum cognitive ability one or more times without performing any physical task to establish one or more maximum cognitive performance baselines, and a second step of measuring the person's maximum physical ability while performing high intensity training one or more times without performing any cognitive task to establish one or more maximum physical performance baselines. A third step can include measuring the person's cognitive and physical dual task abilities while performing high intensity training one or more times, and a fourth step can include an algorithm to compare one or more of the person's maximum cognitive and/or physical single task baselines to a person's cognitive and/or physical dual task high intensity training performance abilities to determine one or more cognitive and/or physical dual task cost performance outputs (e.g., benchmarks) for a person while performing dual task high intensity training. Another step can include prescribing one or more courses of treatment for the person based upon one or more of their cognitive and/or physical dual task cost benchmarks. It will be appreciated that these steps can be used repeatedly and/or in different orders.

Implementations of the disclosed technology can provide a number of advantages such as, but not limited to, the following: the use of fitness and exercise equipment in combination with cognitive stimuli for testing and/or training; the use of activity monitors in combination with cognitive stimuli for testing and/or training; the incorporation of technology to record cognitive performance as a baseline expectation for dual task performance comparison, taking into consideration dual task costs—both with and without technology for physical performance; the comprehensive fitness and cognitive stimuli combination for both wellness and rehabilitation; the incorporation of high intensity training in cognitive stimulation and/or dual task fitness; establishing baseline expectations or normative values, e.g., with age and gender considerations; using analog or digital technology to deliver a cognitive screening assessment designed to make customized dual task intensity recommendations based on an algorithm that allows users to progress to more difficult levels of dual task training; and using the dual task costs to adjust the difficulty of subsequent training.

Implementations of the disclosed technology generally pertain to Dual Task High Intensity Interval Training (DT HIIT), which is a combination of Dual Task (DT) Training and High Intensity Training (HIT). DT training generally includes situations where a person is tested for cognitive performance and physical performance either sequentially or simultaneously. When a person performs cognitive tasks during or after performing physical tasks there is a Dual Task Cost (DTC), which is an impairment of either a person's cognitive and/or physical performance relative to single task performance. HIT generally includes cardiovascular exercise using short periods of intense anaerobic exercise with less intense recovery periods. The disclosed combination of DT training with HIIT is highly effective for accurately describing a person's fitness and predicting a proper course of treatment based thereon. This is especially true for the elderly, for example, who are at risk of falling and for those with degenerative or one-time neurologic conditions such as Parkinson's, multiple sclerosis, stroke, etc.

The cognitive fitness elements may include, but are not limited to, the following: mathematical calculations, following simple or complex (e.g., requiring sequence) commands, object recognition, trivia, short term recall, problem solving, response inhibition, categorization, encoding, retrieval, expressive language, receptive language, story problems, selective attention, sustained attention, visuospatial relations, working memory, speed of response, route finding, serial reasoning, problem solving, quantitative reasoning, verbal reasoning, and other skills in cognition. Cognitive fitness can be measured from a person and can be made from a person in relation to other normative data/expectations. The measurement of such cognitive fitness can be made and recorded by a person's response to a series of cognitive tasks. A software application can be used to measure a person's response to a series of cognitive tasks using variations of the above mentioned categorical cognitive fitness elements, for example.

In certain implementations, sensors may include, but are not limited to, a keyboard, a touch pad, a camera, a microphone, a gyroscope, an accelerometer, or other wearable, portable, or static device. The sensor can send a signal to a software application that would constantly or periodically measure and record a person's cognitive fitness by measuring a variety of a person's responses to cognitive tasks that occur when experiencing and responding to normal activity, such as measuring and recording mathematical calculations, the following of simple or complex (e.g., requiring sequence) commands, object recognition, trivia, short term recall, problem solving, response inhibition, categorization, encoding, retrieval, expressive language, receptive language, story problems, selective attention, sustained attention, visuospatial relations, working memory, speed of response, route finding, serial reasoning, problem solving, quantitative reasoning, verbal reasoning, and other skills in cognition. In this way, a person's cognitive ability can be measured against himself/herself at all times and a record of cognitive ability at all times can be recorded. Scores corresponding to a person's speech patterns, eye movement, and/or ability to maintain balance can be graphed continually and, in certain embodiments, measured against other constant measurements of other people's cognitive finesses. With a certain amount of statistical sampling, characteristics such as age, gender, height, weight, and other specifics to a person can be normalized for more accurate reporting of relative cognitive fitness.

In certain implementations, a specific set of stimuli can be selected for a person or group of people and used to eliminate variability in response and find truer cognitive fitness scores for groups and individuals. Such stimuli can be educational, audiovisual, games, or tests that require significant cognitive response and lend itself to the type of cognition desired to be measured. In its simplest form a person could simply answer questions delivered by a software application.

An assessment of HIIT fitness can only be made from high intensity performance or testing. Quantifiable performance indicators can include various types of recordable data from a workout including, but not limited to, time, heart rate, speed, incline, distance traveled, steps per minute, stride length, symmetry, weight bearing, step length, gait symmetry, perceived exertion, revolutions per minute (RPM), watts, metabolic equivalents (METs), calories, and ground reaction forces. One or more sensors can record data from the training and send a signal to a software application that can constantly or periodically measure and record a person's physical responses to high intensity training. Additionally, performance can be recorded on one device, and manually entered into the program, to start or complete the algorithm.

Dual task costs can be determined for both cognitive and physical abilities at different levels of exertion of either physical or cognitive efforts. For example, a person could have a high impairment of both their cognitive and physical ability that would suggest that they needed to be prescribed a treatment of relatively low levels of dual task activities. Similarly, a low impairment can suggest a treatment of relatively high levels of dual task activities. If either physical or cognitive impairments are relatively disproportionately impaired compared to the other relative to that individual's individual physical or cognitive abilities, this could suggest that the person should focus on the ability (i.e., cognitive or physical) that is more impaired. This sort of asymmetrical dual task cost is the type of impairment that is missed by prior testing methods. Specific physical exercises and specific cognitive skills could be found to be linked as well. Such asymmetrical dual task costs can be determined particularly quickly using HIIT.

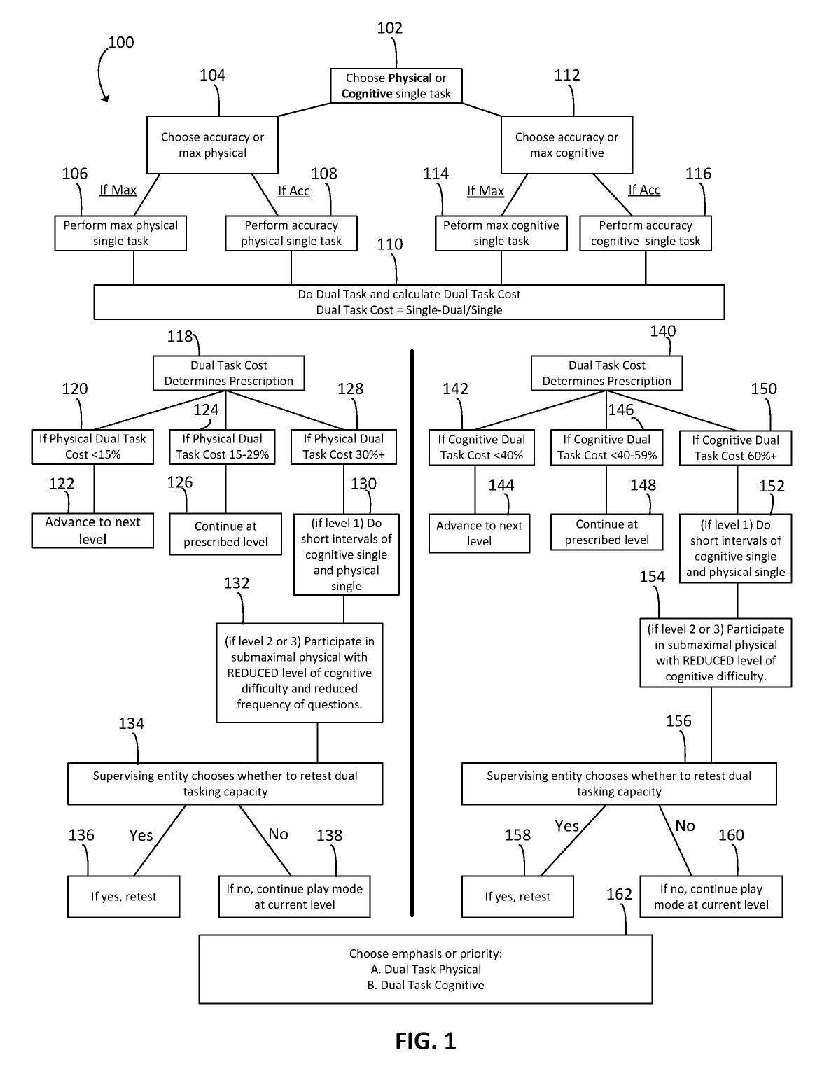

FIG. 1 illustrates an example of a method 100 of using dual task fitness in accordance with certain implementations of the disclosed technology. At 102, a selection is made for a user to perform either a physical single task or a cognitive single task. If the selection is a physical single task, the method 100 proceeds to 104; otherwise, the method 100 proceeds to 112.

At 104, a selection is made between having the user perform a maximum physical single task or a more accurate physical single task. If the selection is maximum physical single task, the method 100 proceeds to 106, where the user performs the maximum physical single task; otherwise, the method 100 proceeds to 108, where the user performs the accuracy physical single task. The method then proceeds to 110.

At 112, a selection is made between having the user perform a maximum cognitive single task or a more accurate cognitive single task. If the selection is maximum cognitive single task, the method 100 proceeds to 114, where the user performs the maximum cognitive single task; otherwise, the method 100 proceeds to 116, where the user performs the accuracy cognitive single task. The method then proceeds to 110.

At 110, a Dual Task Cost (DTC) physical and cognitive benchmarks can be calculated after dual task measure is obtained. The DTC physical benchmark can be calculated by taking the person's maximum physical single task performance, subtracting the dual task physical performance, then dividing the product of that subtraction by the single physical performance, then multiplying the resulting quotient of the prior division by 100. The resulting product is the DTC physical performance output.

The DTC cognitive benchmark can be calculated by taking the person's maximum cognitive single task performance, subtracting the dual task cognitive performance, then dividing the product of that subtraction by the single task performance, then multiplying the resulting quotient of the prior division by 100. The resulting product is the DTC cognitive performance output.

It will be appreciated that the algorithm can be used repeatedly and/or sequentially for combinations of maximum physical single task performance and dual task cost physical performance as well as maximum cognitive single task performance and dual task cost cognitive performance recorded over a period of time to track the person's cognitive and/or physical fitness improvement or deterioration. Thus, both the person's cognitive and physical DTC performance outputs can be determined, monitored, and evaluated.

At 118, the DTC is used in determining/updating a prescription for the user. If the calculated DTC is less than 15%, the method 100 proceeds to 120, where the user is subsequently directed to advance to the next level (at 122); if the calculated DTC is at least 15% but less than 30%, the method 100 proceeds to 124, where the user is directed to continue at the prescribed level (at 126); if the calculated DTC is more than 30%, the method 100 proceeds to 128 and then to 130.

If the user is presently at level 1, he or she is directed to perform short intervals of cognitive single and physical single activities. If the user is at level 2 or 3, the user is directed to participate in submaximal physical activity with a reduced level of cognitive difficulty and reduced frequency of questions (at 132). At 134, a supervising entity (e.g., the system or a person) determines whether to retest the user's dual tasking capacity: if so, the method 100 proceeds to 136 where the user is directed to be re-tested; otherwise, the method 100 proceeds to 138, where the play mode is continued at the current level.

At 140, the DTC is used in determining/updating a prescription for the user. If the calculated DTC is less than 40%, the method 100 proceeds to 142, where the user is subsequently directed to advance to the next level (at 144); if the calculated DTC is at least 40% but less than 60%, the method 100 proceeds to 146, where the user is directed to continue at the prescribed level (at 148); if the calculated DTC is at least 60%, the method 100 proceeds to 150 and then to 152.

If the user is presently at level 1, he or she is directed to perform short intervals of cognitive single and physical single activities. If the user is at level 2 or 3, the user is directed to participate in submaximal physical activity with a reduced level of cognitive difficulty (at 154). At 156, a supervising entity (e.g., the system or a person) determines whether to retest the user's dual tasking capacity: if so, the method 100 proceeds to 158 where the user is directed to be re-tested; otherwise, the method 100 proceeds to 160, where the play mode is continued at the current level.

At 162, a determination is made, based at least in part on the information received from 132 and 154, as to whether a priority should be placed on either dual task physical activity or dual task cognitive activity.

Certain implementations can include measuring a person's maximum cognitive ability one or more times without performing any physical task to establish one or more maximum cognitive performance baselines; measuring the person's maximum physical ability while performing high intensity training one or more times without performing any cognitive task to establish one or more maximum physical performance baselines; measuring the person's cognitive and physical dual task abilities while performing high intensity training one or more times; using an algorithm to compare one or more of the person's maximum cognitive and/or physical single task baselines to a person's cognitive and/or physical dual task high intensity training performance abilities to determine one or more cognitive and/or physical dual task cost performance outputs (benchmarks) for a person while performing dual task high intensity training; and prescribing one or more courses of treatment for the person based upon one or more of their cognitive and/or physical dual task cost benchmarks. It will be appreciated that these steps may be repeated any number of times and further that these steps may be performed in any of a number of suitable orderings.

In certain embodiments, the measurement of a person's maximum cognitive baselines can be done using sensors, signals, or by direct input by way of an electronic device and/or software. For example, the measurement of a person's maximum cognitive baselines can measured by having the person answer a series of questions as quickly as they can. Measurement of the person's maximum cognitive baselines can be measured constantly or periodically with the sensors, signals, and/or direct input by way of an electronic device and/or software.

Measurement of the person's maximum physical baselines can be done using the sensors, signals, and software described above. For example, measurement of one of the person's maximum physical baselines while performing high intensity training can be measured by having the person exert maximum effort while performing high intensity training and recording their performance using any of the standards, sensors, signals, and software described above. Physical performance data can also be entered directly into an electronic device after the physical performance (e.g., read the data from the exercise machine console and enter it into device). Measurement of the person's maximum physical baselines while performing high intensity training can be measured constantly or periodically with the sensors, signals, and/or direct input by way of an electronic device and/or software.

Measurement of the person's cognitive and physical dual task abilities while performing high intensity training can be done using the sensors, signals, devices, and software described above for both cognitive and physical measurements. For example, measurement of the person's cognitive and physical dual task abilities while performing high intensity training can be done by simply having a person perform high intensity training and then having a person answer a series of questions as quickly as they can. There can be several measurements of the person's cognitive and physical dual task abilities while performing high intensity training in order to determine cognitive and physical dual task abilities at different levels of physical exhaustion. For example, measurement of the person's cognitive and physical dual task abilities while performing high intensity training can be measured constantly or periodically using the sensors, signals, devices, and software described above for both cognitive and physical measurements.

The algorithm can use measures of the person's cognitive and physical dual task abilities as inputs and provide as output a measurement of dual task cost cognitive and/or physical performance outputs (e.g., benchmarks) for the person while performing dual task high intensity training.

Over time, the person's cognitive and physical DTC performance outputs can recorded and compared to prior cognitive and physical DTC performance outputs to determine the success or failure of prescribed courses of fitness treatment. Then, users can progress to higher levels of dual task training intensity (e.g., more challenging cognitive tasks and/or more challenging physical tasks). The process of progressing can be turned into a game by allowing the user to earn points and/or progress to higher numerical levels.

In certain embodiments, many measurements of physical and/or cognitive maximum physical single task performance and dual task physical performance can be averaged together over time prior to applying the algorithm and the change in the DTC performance outputs can be graphed over time. Averages of the peaks and troughs of said graph can then be used as average or mean ranges of DTC performance outputs and such average or mean ranges of DTC performance outputs can then be used to determine the course of treatment(s). Certain embodiments may include the use of a cell phone, a personal electronic device such as a Fitbit, or other available sensors, signals, devices, and/or software that can measure the person's cognitive and physical performance. Other embodiments can include a video camera configured to record the person's movements and verbal responses to cognitive and physical stimuli.

Prescribing one or more courses of treatment for the person can based upon one or more of their cognitive and/or physical dual task cost benchmarks or averages thereof. Those prescribing the training can include software, healthcare professionals, personal trainers, athletic trainers, and other suitable entities and/or people. This can provide a simple way for the person to structure his or her goals for a workout. After the initial assessment, users can be directed to engage in one of a number of behaviors. For example, in the Physical realm, the user can attempt to perform at maximal exertion (e.g., watts, calories per minute, speed, RPMs, METs, etc.) or targeted exertion, wherein they are required to match a specified and measurable sub-maximal output (e.g., step length, heart rate, cadence, base of support, RPMs, METs, watts, calories per minute). The user can integrate their physical realm performance with a similar choice on the cognitive realm. Maximal exertion in the cognitive realm can encourage users to get through as many questions, stories, data, or stimuli as possible. Targeted exertion for the cognitive realm can emphasize and, in some instances, reward accuracy in responses, e.g., as measured by percentage correct and/or precision.

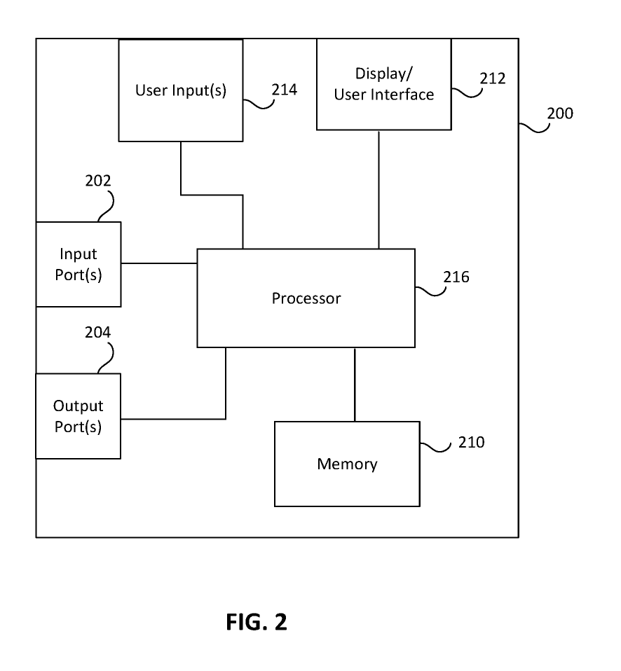

FIG. 2 illustrates an example of a system 200 for using dual task fitness in accordance with certain implementations of the disclosed technology. The system 200 can include one or more input ports 202 and one or more output ports 204 which may each be any suitable electrical signaling medium. The ports 202, 204 may include receivers, transmitters, and/or transceivers. The ports 202, 204 are coupled with one or more processors 216 to process the signals and/or data received at the port(s) 202. Although only one processor 216 is shown in FIG. 2 for ease of illustration, it will be appreciated that multiple processors of varying types may be used in combination, rather than the single processor 216. The resulting output(s) can be stored in a memory 210, as well as displayed on a display 212.

The one or more processors 216 can be configured to execute instructions from memory 210 and may perform any methods and/or associated steps indicated by such instructions, such as displaying values measured to a coupled device according embodiments of the disclosure. Memory 210 may be implemented as processor cache, random access memory (RAM), read only memory (ROM), solid state memory, hard disk drive(s), or any other memory type. Memory 210 can act as a medium for storing data, computer program products, and other instructions.

One or more user inputs 214 are coupled to the processor 216. User input(s) 214 may include a keyboard, mouse, trackball, touchscreen, and/or any other controls employable by a user to interact with the system 200. The display 212 may be a digital screen, a cathode ray tube based display, or any other monitor to display waveforms, measurements, and other data to a user. While the components of the system 200 are depicted as being integrated within a single unit, it will be appreciated that any of these components can be external to each other and can be coupled to each other in any suitable manner (e.g., wired and/or wireless communication media and/or mechanisms). For example, in some embodiments, the display 312 may be remote from the other components.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers. One or more aspects of the disclosure may be embodied in computer-usable data and computer-executable instructions, such as in one or more program modules, executed by one or more computers (including monitoring modules), or other devices. Generally, program modules include routines, programs, objects, components, data structures, etc. that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGA, and the like. Particular data structures may be used to more effectively implement one or more aspects of the disclosure, and such data structures are contemplated within the scope of computer executable instructions and computer-usable data described herein.

The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media. Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Aspects of the present disclosure operate with various modifications and in alternative forms. Specific aspects have been shown by way of example in the drawings and are described in detail herein below. However, it should be noted that the examples disclosed herein are presented for the purposes of clarity of discussion and are not intended to limit the scope of the general concepts disclosed to the specific examples described herein unless expressly limited. As such, the present disclosure is intended to cover all modifications, equivalents, and alternatives of the described aspects in light of the attached drawings and claims.

References in the specification to embodiment, aspect, example, etc., indicate that the described item may include a particular feature, structure, or characteristic. However, every disclosed aspect may or may not necessarily include that particular feature, structure, or characteristic. Moreover, such phrases are not necessarily referring to the same aspect unless specifically noted. Further, when a particular feature, structure, or characteristic is described regarding a particular aspect, such feature, structure, or characteristic can be employed in connection with another disclosed aspect whether or not such feature is explicitly described in conjunction with such other disclosed aspect.

Although specific examples of the invention have been illustrated and described for purposes of illustration, it will be understood that various modifications may be made without departing from the spirit and scope of the invention. Accordingly, the invention should not be limited except as by the appended claims.

We claim:

1. A system, comprising:
at least one sensor configured to obtain a plurality of measurements from a user following a prescribed regimen including physical tasks and cognitive tasks, wherein the plurality of measurements includes at least two measurements selected from a group consisting of the following: a measurement pertaining to a physical single task, a measurement pertaining to a maximum physical single task, a measurement pertaining to a cognitive single task, and a measurement pertaining to a maximum cognitive single task; and
a processor configured to:
calculate a dual task cost physical benchmark based on the plurality of measurements;
calculate a dual task cost cognitive benchmark based on the plurality of measurements;
determine whether to change the prescribed regimen based on the calculated dual task cost physical and cognitive benchmarks.

2. The system of claim 1, wherein changing the prescribed regimen includes advancing from a first level to a second level.

3. The system of claim 1, wherein changing the prescribed regimen includes advancing from a second level to a third level.

4. The system of claim 1, wherein the processor is configured to calculate the dual task cost physical benchmark by taking a maximum physical single task performance by the user, subtracting a dual task physical performance by the user, then dividing the product of the subtraction by the single task physical performance, then multiplying the resulting quotient of the division by 100.

5. The system of claim 4, wherein the processor is further configured to direct the user to advance the prescribed exercise regimen to a next level responsive to the calculated dual task cost physical benchmark being less than 15.

6. The system of claim 4, wherein the processor is further configured to direct the user to maintain the prescribed exercise regimen at a current level responsive to the calculated dual task cost physical benchmark being in a range between 15-30.

7. The system of claim 4, wherein the processor is further configured to direct the user to perform a set of activities responsive to the calculated dual task cost physical benchmark being greater than 30.

8. The system of claim 7, wherein, responsive to a current level of the prescribed regimen being 1, the set of activities includes short intervals of cognitive single tasks and physical single tasks.

9. The system of claim 7, wherein, responsive to a current level of the prescribed regimen being greater than 1, the set of activities includes sub-maximal physical tasks with a reduced level of cognitive difficulty and a reduced frequency of questions presented to the user.

10. The system of claim 1, wherein the processor is configured to calculate the dual task cost cognitive benchmark by taking a maximum cognitive single task performance by the user, subtracting a dual task cognitive performance by the user, then dividing the product of the subtraction by the single task cognitive performance, then multiplying the resulting quotient of the division by 100.

11. The system of claim 10, wherein the processor is further configured to direct the user to advance the prescribed exercise regimen to a next level responsive to the calculated dual task cost cognitive benchmark being less than 40.

12. The system of claim 10, wherein the processor is further configured to direct the user to maintain the prescribed exercise regimen at a current level responsive to the calculated dual task cost cognitive benchmark being in a range between 40-60.

13. The system of claim 10, wherein the processor is further configured to direct the user to perform a set of activities responsive to the calculated dual task cost cognitive benchmark being greater than 60.

14. The system of claim 13, wherein, responsive to a current level of the prescribed regimen being 1, the set of activities includes short intervals of cognitive single tasks and physical single tasks.

15. The system of claim 13, wherein, responsive to a current level of the prescribed regimen being greater than 1, the set of activities includes sub-maximal physical tasks with a reduced level of cognitive difficulty and a reduced frequency of questions presented to the user.

16. The system of claim 1, wherein the processor is further configured to determine whether to have the user re-test his or her dual tasking capacity.

17. The system of claim 1, wherein either or both the at least one sensor and the processor is integrated with a piece of fitness equipment or mobile device such as a smart phone, a tablet computing device, or an electronic wearable device.

18. A method, comprising:
at least one sensor obtaining a plurality of measurements from a user following a prescribed regimen including physical tasks and cognitive tasks, wherein the plurality of measurements includes at least two measurements selected from a group consisting of the following: a measurement pertaining to a physical single task, a measurement pertaining to a maximum physical single task, a measurement pertaining to a cognitive single task, and a measurement pertaining to a maximum cognitive single task;
a processor calculating a dual task cost physical benchmark based on the plurality of measurements;
the processor calculating a dual task cost cognitive benchmark based on the plurality of measurements; and
the processor determining whether to change the prescribed regimen based on the calculated dual task cost physical and cognitive benchmarks.

19. The method of claim 18, wherein calculating the dual task cost physical benchmark includes taking a maximum physical single task performance by the user, subtracting a dual task physical performance by the user, then dividing the product of the subtraction by the single task physical performance, then multiplying the resulting quotient of the division by 100.

20. The method of claim 18, wherein calculating the dual task cost cognitive benchmark includes taking a maximum cognitive single task performance by the user, subtracting a dual task cognitive performance by the user, then dividing the product of the subtraction by the single task cognitive performance, then multiplying the resulting quotient of the division by 100.
